(19) United States
(12) Patent Application Publication
(10) Pub. No.: US 2009/0265115 A1
(43) Pub. Date: Oct. 22, 2009

(54) METHOD AND APPARATUS FOR ANALYZING DNA

(76) Inventor: Mai Wang, San Jose, CA (US)

Correspondence Address: Stevens Law Group, 1754 Technology Drive, Suite #226, San Jose, CA, 95110, US

(21) Appl. No.: 12/391,866

(22) Filed: Feb. 24, 2009

Related U.S. Application Data
(60) Provisional application No. 61/045,511, filed on Apr. 16, 2008.
(60) Provisional application No. 61/102,989, filed on Oct. 6, 2008.

(51) Int. Cl. G06F 19/00; C12Q 1/68

(52) U.S. Cl. 702/19; 435/6

(57) ABSTRACT

This invention is directed to a protocol and design for an apparatus to analyze the distribution and/or ratio of Thymine, Cytosine, Adenine and Guanine of a DNA sequence from a target organism. The result is then used to determine the possible impact the target organism may have in a host such as a human body. The goal is to provide an effective way to diagnose diseases such as infectious diseases, to test the safety of food and drugs, and therefore to create natural and effective solutions for health care and food supply. For example, a DNA analysis method configured according to the invention receives a DNA sequence input and converts it into a reassembled sequence. A result based on the reassembled sequence may then be output.

### RELATED APPLICATIONS

This application claims priority based on U.S. Provisional Patent Application No. 61/045,511, filed on Apr. 16, 2008, entitled “Protocol and Apparatus for Analyzing DNA.” This application also claims priority based on U.S. Provisional Patent Application No. 61/102,989, filed on Oct. 6, 2008, entitled “Protocol and Apparatus for Analyzing DNA (2).”

### BACKGROUND OF THE INVENTION

When an unfamiliar infectious disease surfaces in a society, understanding the disease may likely require the observation of patients throughout the progression of the disease. For example, organizations such as the World Health Organization (WHO) of the United Nations, and the Center for Disease Control and Prevention (CDC) of the United States, may receive reports regarding observed symptoms along with suspected causal links at the outset of an outbreak. Hospitals may continue to report characteristics of the disease, including further symptom developments, prevention techniques, patient responses to various treatment methods, and any results of pathogenic research. Such deeper understanding of the disease is expected to enable health professionals to treat affected patients more effectively and efficiently.

With any given disease, however, this expectation may or may not be realistic within a practical time frame. In other words, the knowledge needed for properly treating the disease may come about only after the disease has spread to a large group of the relevant population. By the time appropriate prevention and treatment methods are defined, it may already be difficult to contain further rampant spreading of the disease.

In the practice of Traditional Chinese Medicine (TCM), practitioners may evaluate the integral physiological state of a patient, based on not only symptoms tangible to the patient, but also various manifestations of the patient's inner physiology. For example, the practitioner may observe the current state of various portions of the patient's tongue and wrist pulse, and test sensitivity as to various meridians (explained below), amongst performing other diagnostic processes. Such diagnosis does not require any labeling of a pathogen or disease, and may be applied regardless of whether the patient's symptoms conform to a known set of symptoms.

Hence, an unfamiliar disease may be treated in accordance with the principles of TCM, even when the patient carries an unrecognized set of symptoms. This approach, however, may require exceptional expertise on the part of the practitioner, which may be a difficult requirement in certain parts of the world, and perhaps even in China. In addition, although this individualistic diagnostic approach is often an advantage in many medical circumstances, it may be inefficient to attack a potentially widespread infectious disease. This is because the effect of the disease on patient A and patient B may be slightly different, because the respective physiologies of patients A and B prior to contracting the disease may be different to begin with. Hence, a TCM practitioner observing the resulting effect on patient A may not necessarily be able to predict with accuracy the resulting effect on patient B.

This patient-specific approach is in fact an advantage of TCM practice, because it allows practitioners to offer each patient an individually tailored treatment method. However, in the case of a new or unfamiliar infectious disease, the immediate goal for society at large is often to contain the disease from spreading amongst the population. Hence, the ability to efficiently determine the effect of a particular pathogen, independent of the particular host (e.g., a human patient), would be advantageous. Such an understanding may allow medical professionals to further determine an appropriate course of treatment quickly before the spread of the disease becomes difficult to contain. As will be seen, the invention provides such a solution in an elegant manner.

Following is a basic overview of some TCM theories, much of which is based on the Yellow Emperor's Classics of Internal Medicine (or Nei Jing). The Nei Jing is the first classic book written in the Warring States period (476-221 BC) of China and it is still the text on which much of today's TCM study is based.

The concept of “Part and Whole” in Nei Jing states that the entire universe is a single entity, and each human being is a part of that entity and therefore contains the same information as the whole entity. TCM practioners adopted this concept a long time ago. For example, they mapped the entire human body to correspond with the inside of a human ear as well as the scalp. Today, Ear Acupuncture and Scalp Acupuncture have become international standards and were included in the California State Board Acupuncture License Exam.

The Five-Element Theory states that there are five dynamic qualities in nature known as the Five Elements: Fire, Water, Wood, Metal and Earth. Everything in the universe has one of the Five Element qualities. The theory describes the “production-subduction” relationships between the Five Elements and their effects on the human body. A summary is illustrated in FIG. 1. Solid arrows represent the “production” relationships whereas the dotted arrows represent the “subduction” relationships.

Traditional Meridian Theory states that there exists a meridian system containing twelve major meridians that run in longitudinal direction within the human body in standing position, along with a meridian network extending from the twelve major meridians throughout the body. The twelve meridians are divided into 3 Yin meridians (Tai-Yin, Shao-Yin and Jue-Yin) and 3 Yang meridians (Tai-Yang, Shao-Yang and Yang-Ming) in both upper and lower limbs (Hand and Foot). Meridians are paths through which energy (also call qi and blood in TCM) travels. Since WHO published the “Standard Acupuncture Nomenclature, 2^{nd} Edition” in 1993, the Meridian Theory and 361 Acupuncture Points have been recognized as international standards.

### BRIEF DESCRIPTION OF THE DRAWINGS

FIG. 1 illustrates a prior art concept.

FIG. 2 illustrates an embodiment of the invention.

FIG. 3 illustrates an embodiment of the invention.

FIGS. 4A-B illustrates an embodiment of the invention.

FIG. 5 illustrates an embodiment of the invention.

FIG. 6 illustrates an embodiment of the invention.

FIG. 7 illustrates an embodiment of the invention.

FIGS. 8A-C illustrates an embodiment of the invention.

FIG. 9 illustrates an embodiment of the invention.

FIG. 10 illustrates an embodiment of the invention.

FIG. 11 illustrates an embodiment of the invention.

### DETAILED DESCRIPTION OF THE INVENTION

This invention is directed to a protocol and design for an apparatus to analyze the distribution and/or ratio of Thymine, Cytosine, Adenine and Guanine of a DNA sequence from a target organism. The result is then used to determine the possible impact the target organism may have in a host such as a human body. The goal is to provide an effective way to diagnose diseases such as infectious diseases, to test the safety of food and drugs, and therefore to create natural and effective solutions for health care and food supply.

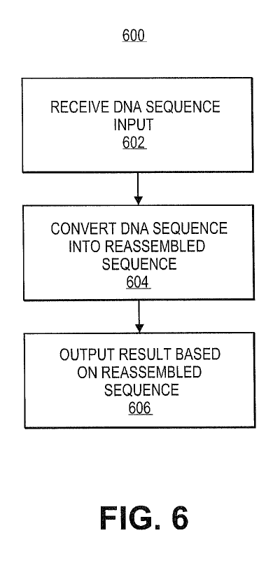

For example, as shown in FIG. 6, a DNA analysis method configured according to the invention receives a DNA sequence input at 602 and converts it into a reassembled sequence at 604. A result based on the reassembled sequence may then be output at 606.

### Foundational Inventive Theories:

Following are theories newly developed within this invention, that serve as the basis for the method and apparatus disclosed. Accuracy of these theories has been demonstrated via the disclosed methods described in the sections below. For more than 30 known infectious diseases, the predicted results matched well with the published information from WHO and CDC.

The Part-and-Whole concept described above may be considered analogous to the mathematical term “Fractal” coined by Benoît Mandelbrot in 1975. Fractal is a system where each part of the whole is a reduced copy of that whole.

Theory of Correspondency: In the context of the Fractal, a “correspondency” exists for two things on the same axis. Thus, when a change occurs in one, the other must be affected as well. For example, suppose A, B, and C are three subsystems within a Fractal System called S. The four-dimensional properties of subsystem A (Xa, Ya, Za, and Ta) are correspondent to the properties of subsystem B (Xb, Yb, Zb, and Tb), the properties of subsystem C (Xc, Yc, Zc, and Tc), and the properties of whole system S (Xs, Ys, Zs, and Ts). If there is any disturbance to Xa, then Xb, Xc, and Xs will all be affected at the same time and the whole system will lose balance. To regain the balance, adjustment must be made at these corresponding positions. It should be noted that time is treated the same way as the other three dimensions. That is, all subsystems are synchronized.

The Concept of Heaven Human and Earth (HUE Concept): The concept of a-three-layer human body was first described in Nei Jing. The human is part of nature and standing in between Heaven and Earth; therefore, he also mimics nature within himself. The three layers are named Heaven, Human, and Earth, where Heaven generally refers to the upper part of the body or the outmost layer covering the whole body; Earth generally refers to the lower part of the body or the most inner layer of the body holding all the internal contents, and Human refers to the layer connecting both Heaven and Earth. Again, based on the Part-and-Whole concept, these three layers also exist in every part of the human body.

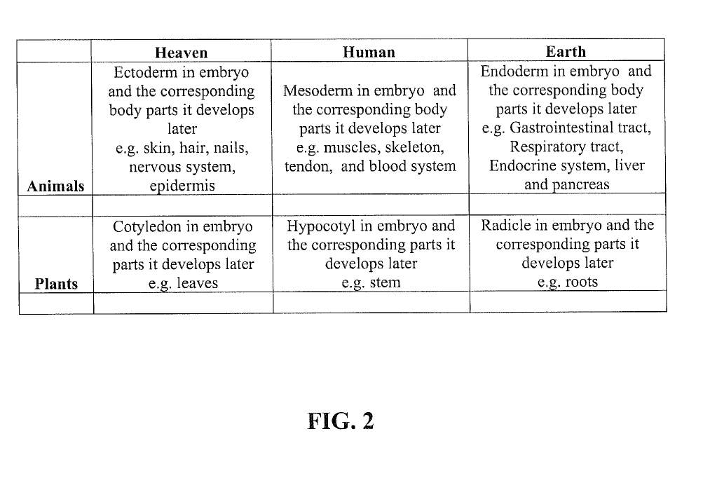

It is proven by modern science that human development starts from three layers of cells called Ectoderm, Mesoderm and Endoderm. The Ectoderm later develops into the outer layer of body such as skin, hair, nails and nervous system. The Mesoderm later develops into the middle layer such as muscle, skeleton, tendons and the circulation system. The Endoderm later develops into the inner layer of the body such as gastrointestinal tract, respiratory tract and the endocrine system. Therefore, we can consider the Heaven layer to include the embryonic Ectoderm and the corresponding developed body parts; the Human layer to include the embryonic Mesoderm and the corresponding developed body parts; and the Earth layer to include the embryonic Endoderm and the corresponding developed body parts. Corresponding body parts of animals and plants are listed in the chart of FIG. 2. Based on the DNA characteristics of the Heaven, Human and Earth layers of the target organism, the physiological effect it might create on a host may be determined.

We can integrate the HUE Concept with the Theory of Correspondency to determine the physiological effects that the target organism may have on its host. The HUE layers of the target organism correspond to the HUE layers of the host. For example, the target organism's Heaven layer will affect the host' Heaven layer, its Human layer will affect the host's Human layer, and its Earth layer will affect the host's Earth layer. Similarly, correspondency also exists between their Five-Element properties. Thus the A, T, C, G and sugar-phosphate of the target organism will directly affect the A, T, C, G and sugar-phosphate of the host. This same theory also applies to the eight phases.

The Four-Period Concept: As a year has four seasons, the life cycle of a natural organism, such as a human, may also be considered to include four periods. The four periods generally entail changes in the internal condition or energy level of that organism. Each period embodies one of the Five-Element properties. For example, the Wood Period is when everything grows, the Fire Period renders the physical body at its strongest state, the Metal Period is when internal energy starts to fall and the body is likely to be dehydrated, and the Water Period is when the body contains least energy during its life cycle. This concept aids practitioners in understanding the internal constitution of the patient in the diagnostic process. For example, a disease causing high fever may be more likely to significantly affect individuals in their Fire Period, rather than those in their Water Period.

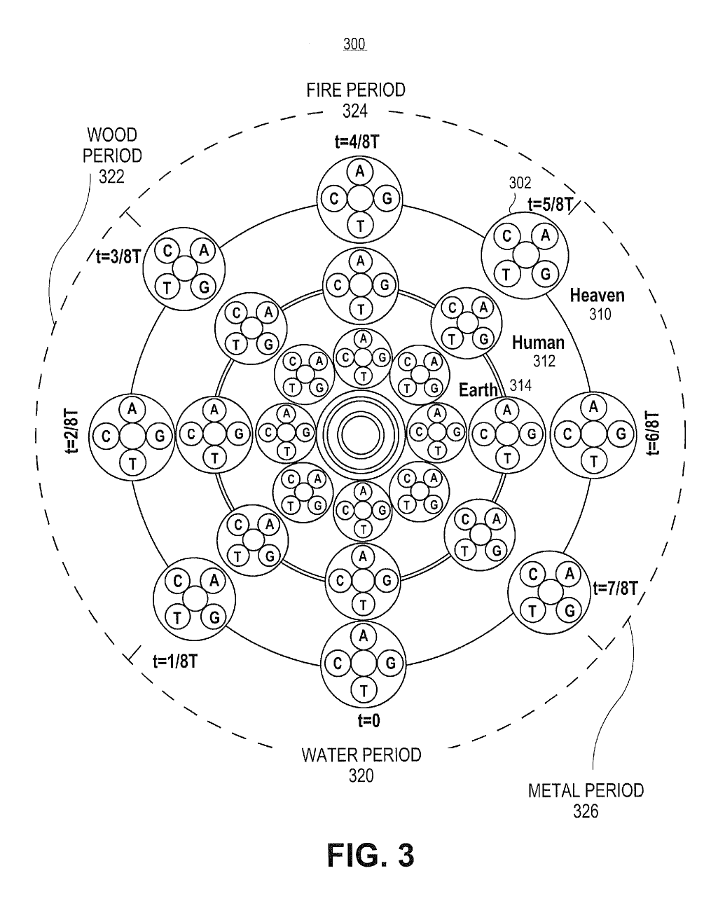

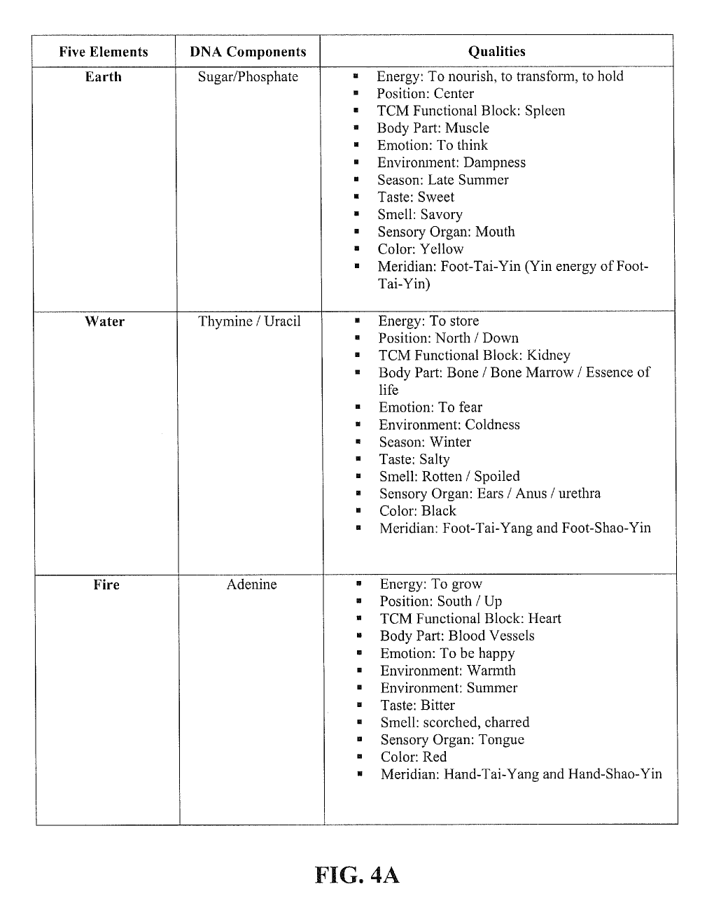

HUE Structure: A HUE Structure, shown in FIG. 3, is a 4×3×8 four-dimensional matrix 300 which exists in every part of the Fractal Universe. “4” stands for the DNA nucleobases corresponding to the Five Elements, as described below, less the Sugar-Phosphate backbone (corresponding to the Earth element). Shown as element 302, it includes a two-dimensional plane where the Fire-Water axis is perpendicular to the Wood-Metal axis, and the Earth element stays in the middle. “3” represents the Heaven 310, Human 312 and Earth 314 layers. “8” corresponds to 8 equal phases in a time domain T. The eight time phases may be considered to include the four periods as described above: Water Period 320, Wood Period 322, Fire Period 324, and Metal Period 326.

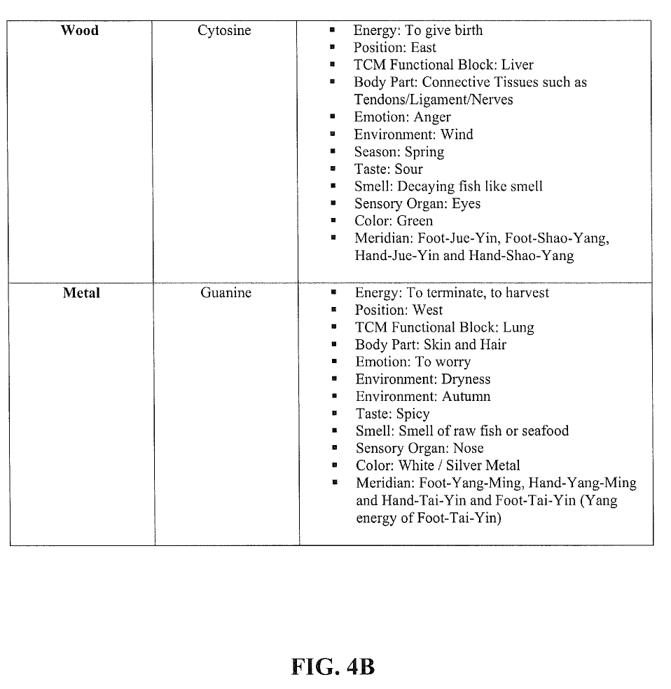

The correspondence between the DNA nucleobases and the properties of the Five Elements are listed in the table of FIGS. 4A-B. Adenine corresponds to Fire because it is energy rich, Thymine to Water because it absorbs energy, Cytosine to Wood because it assists growth, Guanine to Metal because it provides iridescence, and the Sugar-Phosphate backbone to Earth because it nourishes and supports other elements.

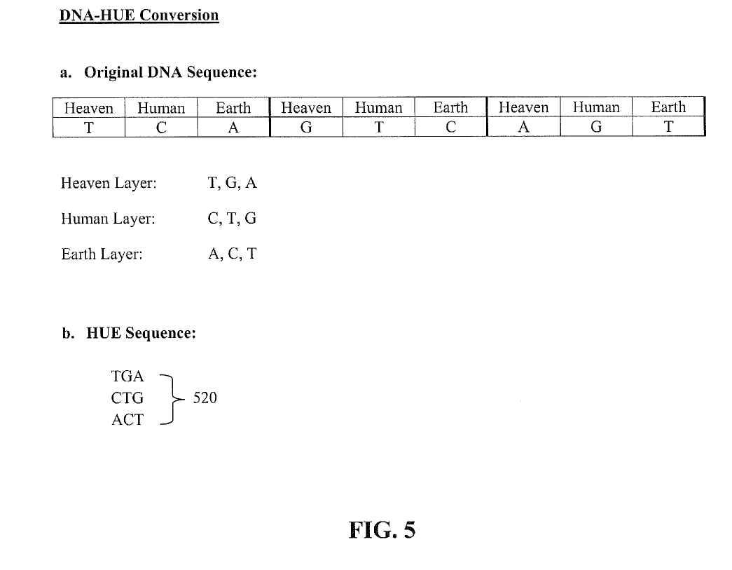

In this invention, a DNA sequence is divided into 3 layers call Heaven, Human and Earth. In the DNA sequence, the Heaven, Human and Earth elements are placed next to each other. FIG. 5 illustrates a conversion from a standard DNA sequence 510 to a reassembled three-layer HUE sequence 520.

### Applications:

Treating and Preventing Diseases. This invention may allow us to understand the exact cause of a disease and predict its development. For example, in the case of an infectious disease, it may reveal the nature of a pathogen and how it is likely to evolve in the human body. This will aid in the effective treatment and prevention of diseases.

Selecting the right food for individuals. This invention may allow the government and consumers to monitor the nature and safety of foods and drugs that we may take in our daily lives. This invention may thus allow an individual to serve as his/her own nutritionist, maintaining health by simply choosing the suitable food from nature. This naturally renders a low-cost, effective health care system.

Effective agricultural planning and food saving. The invention may allow farmers to predict exactly how a plant will grow and what conditions it requires. Farmers can thus build an optimal planting environment and choose the best harvest time. Knowing how energy distributes through a plant (i.e., including leaves, fruits, stems and roots), allows us to make use of the food effectively.

### Method:

The method according to an embodiment of the invention may: (1) map the DNA of a target organism onto a HUE structure, and (2) determine how the target organism may affect its host.

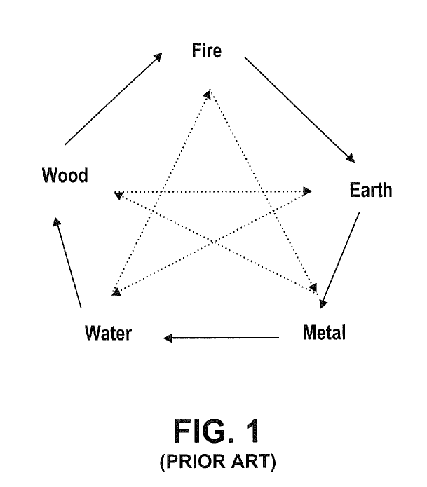

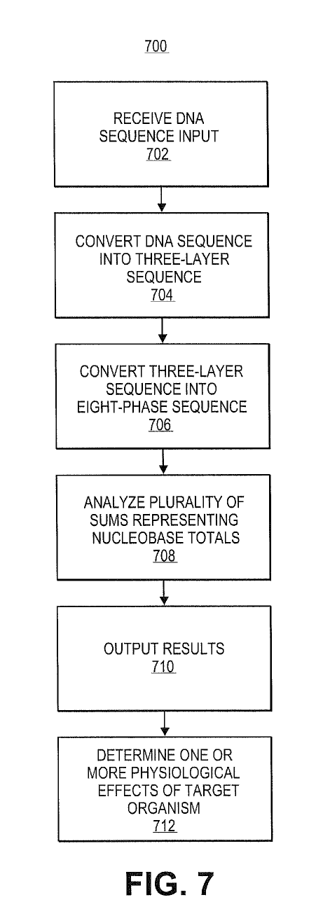

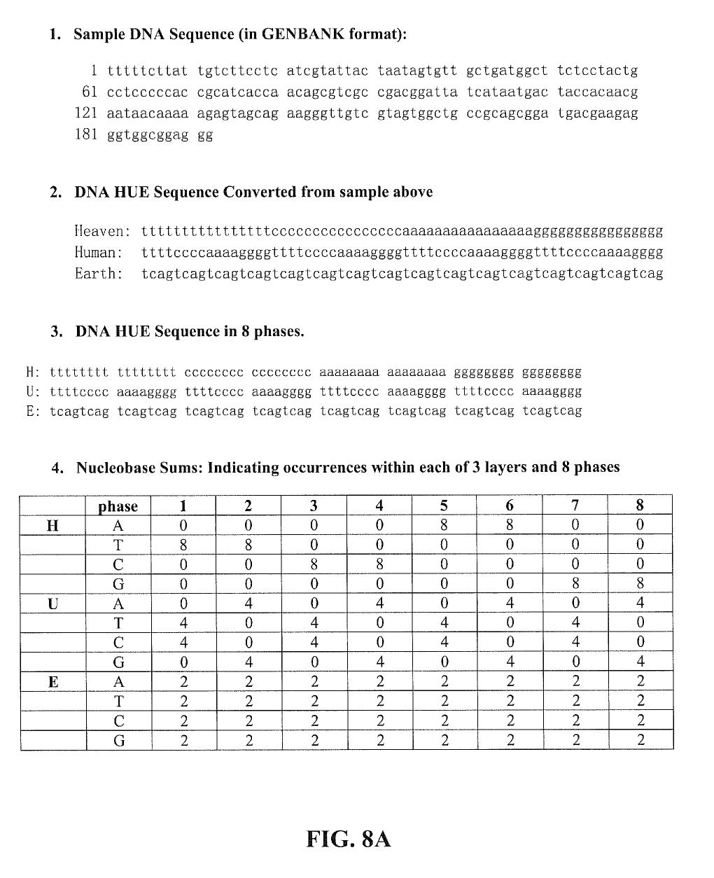

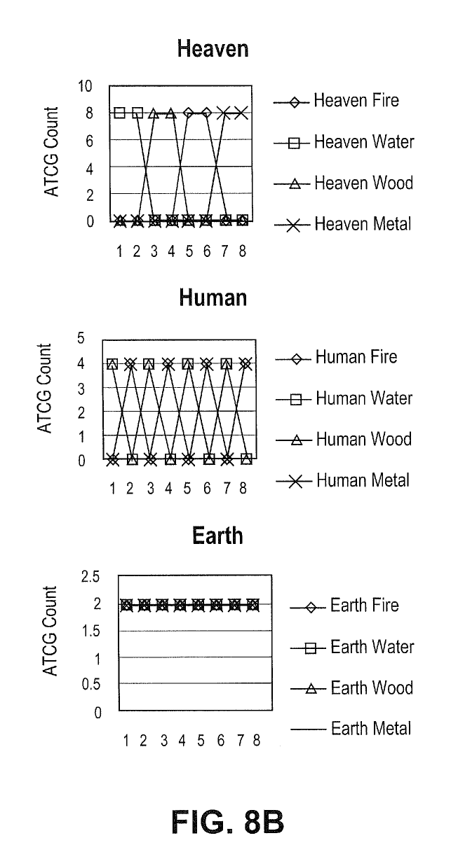

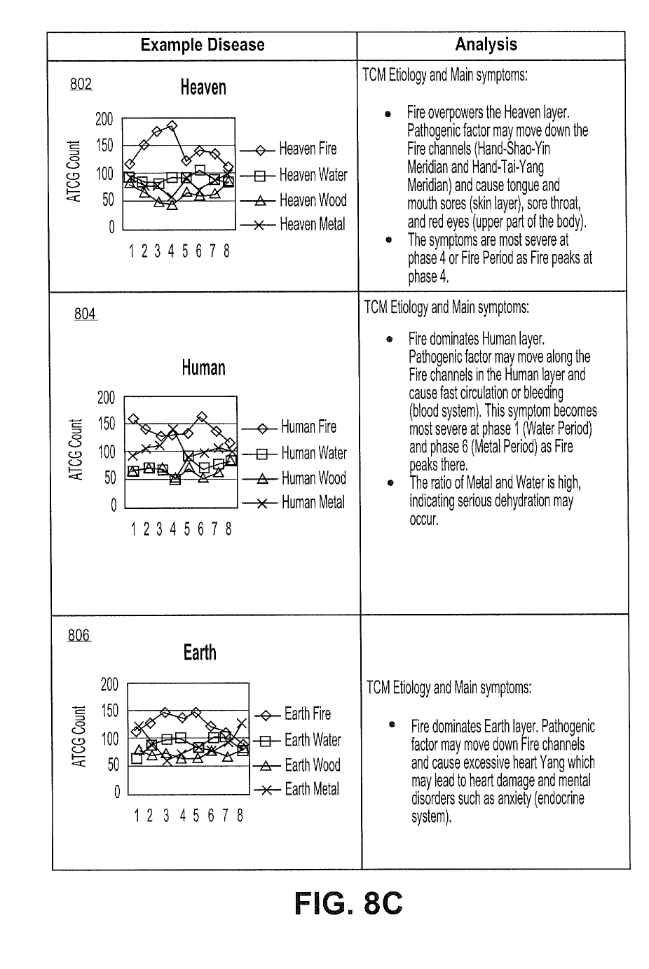

Flowchart 700 of FIG. 7 illustrates a method, to be performed by, for example, a DNA analysis system, configured according to an embodiment of the invention. At 702, a DNA sequence input is received at, for example, an input module. This input may include a digital sequence with nucleobase representations, such as a “ready-made” DNA sequence in GENBANK or FASTA format. Alternatively, actual DNA may be obtained by, for example, extraction from the target organism, after which a DNA code sequence is produced. In the case of a human patient who arrives at a hospital with an unrecognized set of symptoms, a blood sample may be first obtained. Through lab screening of the sample, according to methods known to those of ordinary skill in the art, a foreign organism may be identified. This foreign organism may then be the target organism in question, and its DNA sequence may be extracted. In a preferred embodiment of the invention, the DNA sequence for the complete genome of the target organism is used. A sample DNA sequence in GENBANK format is shown at FIG. 8A-1.

At 704 and 706, the DNA sequence is converted into a reassembled sequence by, for example, a processor. At 704, the DNA sequence may be first converted into a three-layer sequence such as the HUE sequence shown in FIG. 8A-2. In a HUE sequence, a first layer of the three layers includes the first of every three nucleobase representations of the DNA sequence, a second layer of the three layers includes the second of every three nucleobase representations, and a third layer of the three layers includes the third of every three nucleobase representations.

At 706, the three-layer HUE sequence may be divided into eight phases. Each phase occupies ⅛ T of a complete cycle where T is the cycle time. Each of the 8 phases may later be subdivided into smaller units for more detailed analysis. An example of an eight-phase sequence is shown at FIG. 8A-3.

At 708, a plurality of sums may be analyzed, wherein each one of the plurality of sums indicates the total occurrence of, respectively, each one of four nucleobase representations within each of the three layers and eight phases, to produce an analysis result. For example, as shown at FIG. 8A-4, respective sums of each purine Adenine (Fire), Thymine (Water), Cytosine (Wood) and Guanine (Metal) for every layer and phase is determined. The Earth element is not analyzed in this step but can be determined by user based on the Five Element Theory. In the case that multiple DNA sequences are entered, a comparison may be performed. In addition, a ratio in accordance with at least two of the plurality of sums may also be analyzed.

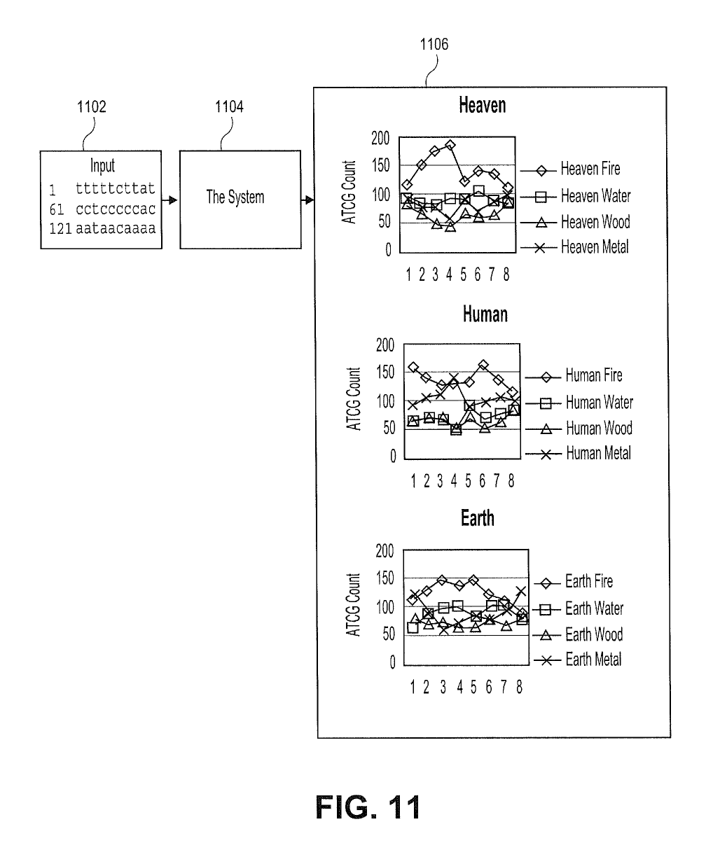

At 710, one or more of the above-described results may be output by, for example, an output module. The results may be displayed in, for example, a visual-friendly form, such as a graph, chart, table, figure, or outline. The output module may display various results, such as a reassembled sequence including either the HUE sequence of FIG. 8A-2 or the 8-phase sequence of FIG. 8A-3, or both. A chart of the plurality of nucleobase sums, such as that of FIG. 8A-4, may be shown as well. A more graphically readable representation of the sums of FIG. 8A-4 is shown in FIG. 8B. Clearly, the DNA sample depicted in FIGS. 8A-B is not a realistic representation of results for a true organism, but rather, serves as an understandable example of the described analysis method. The samples depicted in FIGS. 8C and 11 represent a more realistic picture of a results related to a target organism.

Once the results are output at 710, a practitioner may interpret the results based on both previously known TCM analytical methods, as well as the newly developed theories disclosed herein. Alternatively, at 712, the processor may further determine one or more physiological effects of the target organism, embodying the input DNA sequence, on a host organism, to produce a determination result. This determination result may be based on one or more of a Five-Element Theory, a Meridian Theory, a Theory of Correspondency, a Concept of Human Heaven and Earth, and a Four-Period Concept as described above. For example, FIG. 8C illustrates an analysis of the results based on steps 702-710 above. As shown in the Heaven 802, Human 804, and Earth 806 sections, such interpretation differentiates findings amongst the three layers. For example, the findings in regard to the Heaven layer correlate to symptoms at the skin layer/upper part of the body; the findings in regard to the Human layer correlate to symptoms of the blood system; and the findings in regard to the Earth layer correlate to the symptoms of the endocrine system. These correlations are consistent with those described in FIG. 2 and the above paragraphs. These interpretive results, as well as the criteria on which the interpretations are based, may be output in display form.

As will be understood by one of ordinary skill in the art, the results will aid a health practitioner in understanding the characteristics of the disease, and thus in attacking the progression of the disease, according to the principles of TCM and the newly developed theories described herein.

### Apparatus:

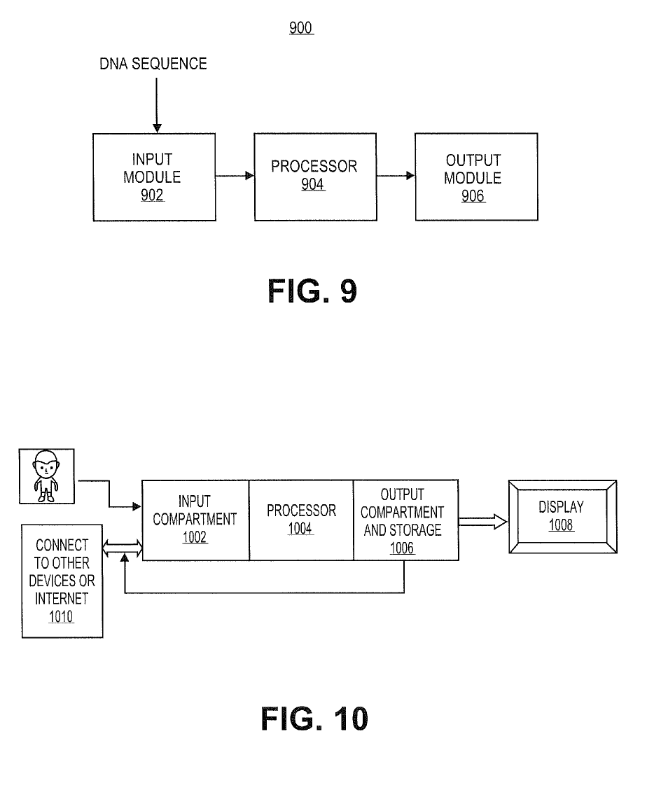

In an embodiment of the invention as shown in FIG. 9, a DNA analysis system for performing a method as described above may comprise an input module 902 to receive a DNA sequence input, a processor 904 to convert the DNA sequence into a reassembled sequence; and an output module 906 to output a result based on the reassembled sequence.

Another embodiment of a DNA analysis system is shown in FIG. 10. The input compartment 1002 may receive a a DNA sequence from any source, including directly from the target organism or from an outside storage such as via an internet connection at 1010. The system includes processor 1004 to perform the processes described above. The output compartment 1006 may include a storage unit to save results, and may output results to display 1008. The output compartment may also allow the user to output results to another device via the external connection at 1010.

In an embodiment of the invention, the system may allow the user to enter multiple DNA sequences for comparison. The system may also include software running from a host computer or a module that is embedded inside a multi-functional device.

FIG. 11 illustrates an input DNA sequence 1102 entering the DNA analysis system 1104, which displays HUE chart results 1106.

The invention may also involve a number of functions to be performed by a computer processor, such as a microprocessor. The microprocessor may be a specialized or dedicated microprocessor that is configured to perform particular tasks according to the invention, by executing machine-readable software code that defines the particular tasks embodied by the invention. The microprocessor may also be configured to operate and communicate with other devices such as direct memory access modules, memory storage devices, Internet related hardware, and other devices that relate to the transmission of data in accordance with the invention. The software code may be configured using software formats such as Java, C++, XML (Extensible Mark-up Language) and other languages that may be used to define functions that relate to operations of devices required to carry out the functional operations related to the invention. The code may be written in different forms and styles, many of which are known to those skilled in the art. Different code formats, code configurations, styles and forms of software programs and other means of configuring code to define the operations of a microprocessor in accordance with the invention will not depart from the spirit and scope of the invention.

Within the different types of devices, such as laptop or desktop computers, hand held devices with processors or processing logic, and also possibly computer servers or other devices that utilize the invention, there exist different types of memory devices for storing and retrieving information while performing functions according to the invention. Cache memory devices are often included in such computers for use by the central processing unit as a convenient storage location for information that is frequently stored and retrieved. Similarly, a persistent memory is also frequently used with such computers for maintaining information that is frequently retrieved by a central processing unit, but that is not often altered within the persistent memory, unlike the cache memory. Main memory is also usually included for storing and retrieving larger amounts of information such as data and software applications configured to perform functions according to the invention when executed by the central processing unit. These memory devices may be configured as random access memory (RAM), static random access memory (SRAM), dynamic random access memory (DRAM), flash memory, and other memory storage devices that may be accessed by a central processing unit to store and retrieve information. The invention is not limited to any particular type of memory device, or any commonly used protocol for storing and retrieving information to and from these memory devices respectively.

The methods and systems disclosed herein include a novel approach for understanding, treating, and preventing previously unrecognized infectious diseases, as well as the effects of various organisms on various hosts in general. However, the scope of the invention extends to other applications where such functions are useful. Furthermore, while the foregoing description has been with reference to particular embodiments of the invention, it will be appreciated that these are only illustrative of the invention and that changes may be made to those embodiments without departing from the principles of the invention, the scope of which is defined by the appended claims and their equivalents.

What is claimed is:

1. A DNA analysis system, comprising:
an input module to receive a DNA sequence input;
a processor to convert the DNA sequence into a reassembled sequence;
an output module to output a result based on the reassembled sequence.

2. The DNA analysis system of claim 1, wherein the result output includes the reassembled sequence.

3. The DNA analysis system of claim 1, wherein the reassembled sequence includes three layers.

4. The DNA analysis system of claim 3, wherein a first layer of the three layers includes the first of every three nucleobase representations of the DNA sequence, a second layer of the three layers includes the second of every three nucleobase representations, and a third layer of the three layers includes the third of every three nucleobase representations.

5. The DNA analysis system of claim 3, wherein the processor further divides the reassembled sequence into eight phases.

6. The DNA analysis system of claim 5, wherein each of the eight phases represents 1/8 of a complete time cycle.

7. The DNA analysis system of claim 5, wherein the processor further divides the reassembled sequence into further subdivided phases within each of the eight phases.

8. The DNA analysis system of claim 5, wherein the processor further analyzes a plurality of sums, wherein each one of the plurality of sums indicates the total occurrence of, respectively, each one of four nucleobase representations within each of the three layers and eight phases, to produce an analysis result.

9. The DNA analysis system of claim 8, wherein the processor further analyzes a ratio in accordance with at least two of the plurality of sums.

10. The DNA analysis system of claim 8, wherein the result output includes the analysis result.

11. The DNA analysis system of claim 1, wherein the processor further determines a physiological effect of a target organism, embodying the input DNA sequence, on a host organism, to produce a determination result.

12. The DNA analysis system of claim 11, wherein the determination result is based on one of a Five-Element Theory, a Meridian Theory, a Theory of Correspondency, a Concept of Human Heaven and Earth, and a Four-Period Concept.

13. The DNA analysis system of claim 11, wherein the result output includes the determination result.

14. The DNA analysis system of claim 11, wherein the result output includes criteria on which the determination result is based.

15. The DNA analysis system of claim 1, wherein the result output includes one of a graph, chart, table, figure, and outline.

16. A DNA analysis method, comprising:
receiving a DNA sequence input;
converting the DNA sequence into a reassembled sequence;
outputting a result based on the reassembled sequence.

17. The DNA analysis method of claim 16, wherein the result output includes the reassembled sequence.

18. The DNA analysis method of claim 16, wherein the reassembled sequence includes three layers.

19. The DNA analysis method of claim 18, wherein a first layer of the three layers includes the first of every three nucleobase representations of the DNA sequence, a second layer of the three layers includes the second of every three nucleobase representations, and a third layer of the three layers includes the third of every three nucleobase representations.

20. The DNA analysis method of claim 18, further comprising:
dividing the reassembled sequence into eight phases.

21. The DNA analysis method of claim 20, wherein each of the eight phases represents ⅛ of a complete time cycle.

22. The DNA analysis method of claim 20, further comprising:
dividing the reassembled sequence into further subdivided phases within each of the eight phases.

23. The DNA analysis method of claim 20, further comprising:
analyzing a plurality of sums, wherein each one of the plurality of sums indicates the total occurrence of, respectively, each one of four nucleobase representations within each of the three layers and eight phases, to produce an analysis result.

24. The DNA analysis method of claim 23, further comprising:
analyzing a ratio in accordance with at least two of the plurality of sums.

25. The DNA analysis method of claim 23, wherein the result output includes the analysis result.

26. The DNA analysis method of claim 16, further comprising:
determining a physiological effect of a target organism, embodying the input DNA sequence, on a host organism, to produce a determination result.

27. The DNA analysis method of claim 26, wherein the determination result is based on one of a Five-Element Theory, a Meridian Theory, a Theory of Correspondency, a Concept of Human Heaven and Earth, and a Four-Period Concept.

28. The DNA analysis method of claim 26, wherein the result output includes the determination result.

29. The DNA analysis method of claim 26, wherein the result output includes criteria on which the determination result is based.

30. The DNA analysis method of claim 16, wherein the result output includes one of a graph, chart, table, figure, and outline.

31. A computer-readable storage medium that stores computer instructions which, when executed by a computer, cause the computer to perform operations comprising:
receiving a DNA sequence input;
converting the DNA sequence into a reassembled sequence;
outputting a result based on the reassembled sequence.

32. The computer-readable storage medium of claim 31, wherein the result output includes the reassembled sequence.

33. The computer-readable storage medium of claim 31, wherein the reassembled sequence includes three layers.

34. The computer-readable storage medium of claim 33, wherein a first layer of the three layers includes the first of every three nucleobase representations of the DNA sequence, a second layer of the three layers includes the second of every three nucleobase representations, and a third layer of the three layers includes the third of every three nucleobase representations.

35. The computer-readable storage medium of claim 33, further storing instructions which, when executed by a computer, cause the computer to perform operations comprising:
dividing the reassembled sequence into eight phases.

36. The computer-readable storage medium of claim 35, wherein each of the eight phases represents ⅛ of a complete time cycle.

37. The computer-readable storage medium of claim 35, further storing instructions which, when executed by a computer, cause the computer to perform operations comprising:
dividing the reassembled sequence into further subdivided phases within each of the eight phases.

38. The computer-readable storage medium of claim 35, further storing instructions which, when executed by a computer, cause the computer to perform operations comprising:
analyzing a plurality of sums, wherein each one of the plurality of sums indicates the total occurrence of, respectively, each one of four nucleobase representations within each of the three layers and eight phases, to produce an analysis result.

39. The computer-readable storage medium of claim 38, further storing instructions which, when executed by a computer, cause the computer to perform operations comprising:
analyzing a ratio in accordance with at least two of the plurality of sums.

40. The computer-readable storage medium of claim 38, wherein the result output includes the analysis result.

41. The computer-readable storage medium of claim 31, further storing instructions which, when executed by a computer, cause the computer to perform operations comprising:
determining a physiological effect of a target organism, embodying the input DNA sequence, on a host organism, to produce a determination result.

42. The computer-readable storage medium of claim 41, wherein the determination result is based on one of a Five-Element Theory, a Meridian Theory, a Theory of Correspondency, a Concept of Human Heaven and Earth, and a Four-Period Concept.

43. The computer-readable storage medium of claim 41, wherein the result output includes the determination result.

44. The computer-readable storage medium of claim 41, wherein the result output includes criteria on which the determination result is based.

45. The computer-readable storage medium of claim 31, wherein the result output includes one of a graph, chart, table, figure, and outline.
